(19) United States
(12) Plant Patent Application Publication
(10) Pub. No.: US 2008/0148438 P1
(43) Pub. Date: Jun. 19, 2008

(54) Lemon tree named "Eureka Seedless"

(76) Inventor: Hannes Breedt, Limburg (ZA)

Correspondence Address: STRATTON BALLEW, 213 S 12TH AVE, YAKIMA, WA, 98902, US

(21) Appl. No.: 11/641,610

(22) Filed: Dec. 18, 2006

(51) Int. Cl. A01H 5/00

(52) U.S. Cl. PLT/201

(57) ABSTRACT

A new lemon tree (Citrus limon) named ‘Eureka Seedless’ is disclosed. The new variety is similar in many respects to its parent ‘Eureka,’ but is distinguishable from ‘Eureka’ by its consistently seedless fruit.

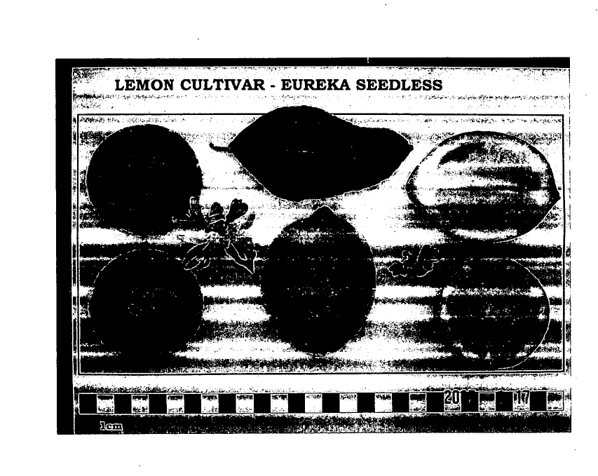

### CROSS-REFERENCE TO RELATED APPLICATIONS

None

### STATEMENT REGARDING FEDERALLY SPONSORED RESEARCH OR DEVELOPMENT

None

### LATIN NAME OF THE GENUS AND SPECIES OF THE PLANT CLAIMED

Citrus limon

### VARIETY DENOMINATION

‘Eureka Seedless’

### BACKGROUND AND SUMMARY OF THE INVENTION

The ‘Eureka Seedless’ lemon tree originated as a limb sport mutation of a ‘Eureka’ lemon tree (not patented), discovered in 1996 in Hoedspruit, Mpumalanga, South Africa. The new variety was notable for its production of seedless fruit, and was selected for propagation and observation. ‘Eureka Seedless’ was first asexually propagated in 2000 at the Institute for

Tropical and Subtropical Crops at Neslpruit, South Africa, using tip grafting methods. The new variety has been observed to retain its distinctive characteristics over successive asexually propagated generations.

### BRIEF DESCRIPTION OF THE DRAWINGS

The accompanying color photographic illustration shows typical specimens of the foliage, flowers and fruit of the new lemon tree.

### DETAILED DESCRIPTION OF THE NEW VARIETY

The following is a detailed botanical description of the ‘Eureka Seedless’ lemon tree, based on observations of specimens grown near Nelspruit, South Africa. All color terminology, except those colors described in common terms, is set forth in accordance with The Royal Horticultural Society Colour Chart.

It should be understood that the botanical characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Tree:
Medium height (usually 8 to 12 feet depending on cultural conditions), spreading, precocious, productive: No differences noted between ‘Eureka Seedless and standard ‘Eureka.’
Young shoots:
   Anthocyanin coloration.—Present, intense.
Leaves:
   Length.—102 mm, width 59 mm, ovate, upfolded, base oblique, tip acute, margin crenulate. Petiole length 11 cm, thickness 3 mm, color yellow-green 144B.
Flowers:
   Typical of standard ‘eureka.’—Color dark pink to white.
Fruit:
   Clustering of fruits.—Predominantly absent.
   Size.—Length 93 mm, diameter 67 mm, length/diameter ratio 1.39.
   Position of broadest part.—In middle.
   Shape of proximal part.—Rounded.
   Neck.—Present, length 8.8 mm.
   Shape of distal part.—Rounded.
   Nipple.—Present, prominent, length 6.5 mm.
   Stylar scar.—Present, small, 1.5 mm.
   Radial grooves at distal end.—Absent or weakly expressed.
   Fruit surface.—Color yellow-green 144A, low gloss, moderate texture, pitting present, more prominent than pebbling.
   Fruit rind.—Thickness 4.0 mm.
   Fruit flesh color.—Light yellow green.
   Fruit core.—Diameter 3.9 mm.
   Rudimentary segments.—Absent.
   Well-developed segments.—Usually about 9.
   Seeds.—None.
Use: Fresh market, export
Keeping quality: Stores well, similar to ‘Eureka’

What is claimed is:

1. A new and distinct lemon tree substantially as shown and described herein.
